(19) United States
(12) Patent Application Publication
(10) Pub. No.: US 2007/0280890 A1
(43) Pub. Date: Dec. 6, 2007

(54) System and method for disinfecting and drying a surface

(76) Inventor: Joseph Frank Gravlee, Cherokee, AL (US)

Correspondence Address: BRINKS HOFER GILSON & LIONE, P.O. BOX 10395, CHICAGO, IL, 60610, US

(21) Appl. No.: 11/443,328

(22) Filed: May 30, 2006

(51) Int. Cl. A61K 9/14; A61K 31/13

(52) U.S. Cl. 424/46; 514/673

(57) ABSTRACT

Disinfectant systems and methods of disinfecting a surface for drying are disclosed. One system comprises a carrier for drying the surface and an anti-microbial component for disinfecting the surface. The ant-microbial component forms a film-free coating containing an organic halogen salt when the carrier evaporates from the surface.

### FIELD OF THE INVENTION

The present invention relates to systems and methods of disinfecting and drying a surface. More particularly, the present invention relates to systems and methods of preparing a surface for treatment.

### BACKGROUND OF THE INVENTION

There are a number of ways of preparing an organic or inorganic surface prior to treating or bonding to the surface. For example, a hoof surface of a mammal is typically cleaned and prepared with a layer of primer that leaves a residual film, adhering to the hoof surface. An adhesive layer is then applied on the residual film opposite the hoof surface to bond a second surface thereto. However, the strength of such bond is typically dependent on the integrity of the primer residual film on the hoof surface. Thus, a shoe surface that is mounted on the adhesive layer opposite the hoof surface also relies on the integrity of the primer residual film to bond a shoe to the hoof. Moreover, bacteria caught or microbial growth between the surfaces lessen the bond strength and reduce the bond life of the surfaces. Additionally, moisture caught between the surfaces further reduces the bond between the surfaces.

Thus, there is a need to provide a way of disinfecting and drying surfaces in preparation of various treatments thereto such as bonding.

### BRIEF SUMMARY OF THE INVENTION

The present invention generally provides systems and methods of disinfecting and drying surfaces in preparation for various treatments such as bonding. Embodiments of the present invention greatly reduce the bacterial population on a surface. The embodiments of the present invention also enhance the bonding performance of the surface by reducing or eliminating moisture from the surface. This is accomplished by leaving a film-free coating on the surface when a drying agent of a system of the present invention evaporates from the surface, avoiding formation of a moisture barrier film. Without such moisture barrier film, bonding on the surface is enhanced even when the surface comprises inorganic material. Additionally, the present invention prevents seeding of microbes that may grow on the surface thereafter. Such microbes are known to potentially cause detachment from surfaces bonded to one another. Furthermore, due to the disinfecting and drying features of the system, embodiments of the invention may be applied to treatment of wounds.

In one embodiment, the present invention provides a disinfectant system for drying a surface. The system comprises an aerosol carrier for drying a surface and an anti-microbial compound for disinfecting the surface. The compound has a component that leaves a film-free coating containing an organic halogen salt on the surface when the carrier evaporates from the surface.

In another embodiment, the present invention provides a disinfectant system for preparing a surface for treatment. In this embodiment, the system comprises an organic drying agent preferably as an aerosol for drying the surface and an anti-microbial compound for disinfecting the surface. In this embodiment, the compound has an organic iodine compound that leaves a film-free halogen salt coating on the surface when the drying agent evaporates from the surface.

In yet another embodiment, the present invention provides a disinfectant system for preparing a hoof of a horse prior to bonding. The system comprises an organic component for drying the surface and an anti-microbial component for disinfecting the surface. The anti-microbial component comprises ethylenediamine dihydriodide to leave a film-free halogen salt coating on the surface when the organic component evaporates from the surface.

In another example, the present invention provides a method of disinfecting a surface for drying. The method comprises applying a disinfectant system on the surface. The disinfectant system comprises a carrier component for drying the surface and an anti-microbial component for disinfecting the surface. The anti-microbial component has a halogen compound which remains as a film-free coating on the surface when the carrier evaporates.

In another example, the present invention provides a method of preparing a surface for treatment. In this example, the method comprises applying a disinfectant system comprised of an organic drying agent to dry the surface and an anti-microbial compound to disinfect the surface. The anti-microbial compound is an organic iodine compound which remains as a halogen salt coating on the surface when the drying agent evaporates.

In yet another example, the present example provides a method of preparing a hoof surface of a horse for bonding. In this example, the method comprises disinfecting the surface with a disinfectant system with a volatile organic component and an anti-microbial component. The anti-microbial is ethylenediamine dihydriodide. The method further includes drying the surface with the volatile organic component. This prepares for enhanced bonding on the hoof surface.

Further objects, features, and advantages of the present invention will become apparent from consideration of the following description and the appended claims when taken in connection with the accompanying drawings.

### BRIEF DESCRIPTION OF THE DRAWINGS

FIG. 1 is a flow chart depicting a method of disinfecting a surface for drying in accordance with one example of the present invention; and

FIG. 2 is a flow chart depicting a method of preparing a surface for treatment in accordance with another example of the present invention.

### DETAILED DESCRIPTION OF THE INVENTION

Embodiments of the present invention generally provide systems and methods of disinfecting and drying a surface. The embodiments provide a carrier, preferably an aerosol, for drying a surface and an anti-microbial compound for disinfecting the surface. When the carrier evaporates to dry the surface, a film-free coating is left on the surface. Due to the film-free coating on the surface, further treatments such as bonding the surface to another organic or inorganic surface are enhanced.

In one embodiment, the present invention provides a disinfectant system for drying a surface. The system comprises a carrier for drying the surface and an anti-microbial compound for disinfecting the surface. In this embodiment, the carrier is a drying agent that is preferably an organic compound or a volatile organic compound comprising a drying agent. The drying agent may comprise an ether-based compound. Ether, which is also called diethyl ether, diethyl oxide, ethyl ether, and ethoxyethane, has the chemical formula C₂H₅OC₂H₅. At standard temperature and pressure ether is a colorless liquid, with a relatively sweet smell. Ether is a relatively volatile organic compound and in air its vapor can form an explosive mixture. The boiling point of ether is 94° F. (34.5° C.).

At ambient conditions, the carrier evaporates from the surface on which the disinfectant system is applied. In one embodiment, the drying agent evaporates from the surface, leaving a film-free coating as discussed below. In this embodiment, the drying agent may be a mixture of between about 20% and 80% by weight methyl nonafluoroisobutyl ether and between about 20% and 80% by weight methyl nonafluorobutyl ether. Preferably, the mixture of methyl nonafluoroisobutyl ether and methyl nonafluorobutyl ether mixture may be obtained from the 3M Company having a product name HFE-7100 3M Novec™ engineered fluid. The film-free coating allows for enhanced bonding, for example, since there is no layer between the bonding surfaces. Thus, the bonding surfaces are allowed more surface area contact for enhanced bonding.

The anti-microbial preferably has a halogen component. The halogen component may comprise an organic halogen compound of fluorine, chlorine, bromine, or iodine. Preferably, the halogen component is an organic iodine compound. In this embodiment, the film-free coating contains an organic halogen salt on the surface when carrier evaporates from the surface. The film-free coating allows for increased surface area contact between bonding surfaces to enhance bonding or adhevisivity therebetween.

In this embodiment, the organic iodine compound contains ethylenediamine dihydriodide (or “EDDI”). Other names for ethylenediamine dihydriodide include dthylene diamine dihydriodide; organic iodine; Ethylenediamine dihydroiodide; Dihydroiodide Ethanediamine; Ethylendiammoniumdiiodid; Diioduro de etilendiamonio; 1,2-Diaminoethane; and Dihydroiodide Ethanediamine. In this embodiment, the EDDI is in a crystalline form. Moreover, the system is preferably provided in an aerosol. However, it is understood that any other anti-microbial may be used that leaves a film free coating as the carrier evaporates. For example, the anti-microbial may include sodium hypochlorite, NaOCl or a bromine compound.

In one embodiment, the carrier comprises between about 70 and 98 weight percent and the anti-microbial comprises between about 2 and 30 weight percent of the system. Preferably, the carrier comprises between about 80 and 95 weight percent and the anti-microbial comprises between about 5 and 20 weight percent of the system. More preferably, the carrier comprises about 90 and the anti-microbial compound comprises about 10 weight percent of the system.

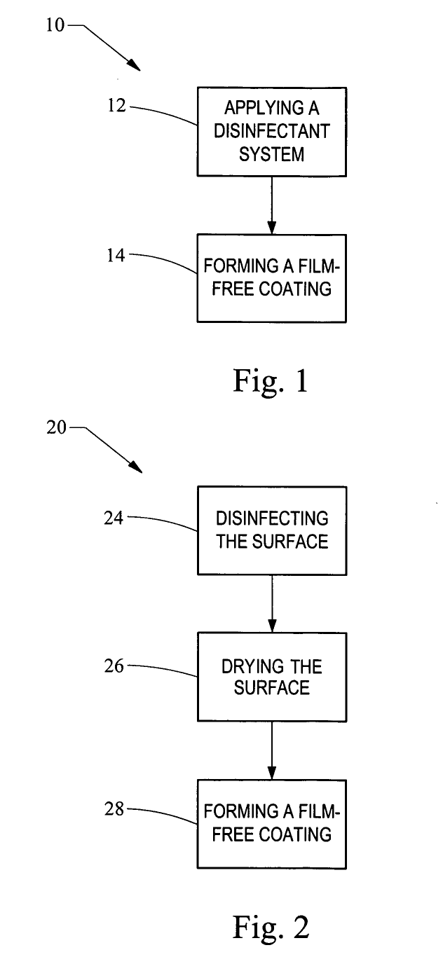

FIG. 1 illustrates a method 10 of disinfecting a surface for drying in accordance with one example of the present invention. As shown, the method 10 comprises applying in box 12 the disinfectant system on the surface to be dried and disinfected. As mentioned above, the disinfectant system comprises the carrier for drying and the anti-microbial compound for disinfecting the surface. The anti-microbial compound is preferably an organic iodine compound, e.g., EDDI. The anti-microbial compound reduces the bacterial population on the surface and also prevents seeding of microbial microbes that may grow thereafter. Thus, in a situation where the surface to which the disinfectant system is applied is a bonding surface, the anti-microbial compound prevents subsequent detachment from the bonding surface.

FIG. 1 depicts the method further comprising leaving or forming a film-free coating in box 14. The film-free coating enhances bonding on the surface applied with the disinfectant system. This is accomplished by the evaporation of the carrier from the surface. In this example, the carrier comprises an ether based compound which evaporates substantially on contact at ambient conditions, leaving the film-free coating. In this embodiment, the drying agent may be a mixture of between about 20% and 80% by weight methyl nonafluoroisobutyl ether and between about 20% and 80% by weight methyl nonafluorobutyl ether. Preferably, the mixture of methyl nonafluoroisobutyl ether and methyl nonafluorobutyl ether mixture may be obtained from the 3M Company having a product name HFE-7100 3M Novec™ engineered fluid. The film-free coating comprises an organic crystalline halogen salt on the surface. Without a film or any type of barrier on the surface, the bonding properties on the surface are enhanced.

In one example, the disinfectant system may be used as a pre-bonding application to a hoof of a mammal, e.g., a horse. In this example, the disinfectant system may be used to prepare for bonding patches, pads, or rigid surfaces such as hoofs. FIG. 2 illustrates a method 20 of preparing a hoof surface of a mammal for bonding in accordance with another example of the present invention. In this example, a bottom surface of a mammal's hoof is cleaned, and the disinfectant system is applied thereon. The disinfectant system comprises the volatile organic compound and the anti-microbial compound both mentioned above. As shown in box 24, the anti-microbial compound disinfects the hoof surface. In this example, the anti-microbial compound comprises EDDI. The anti-microbial compound serves to reduce the bacteria population on the hoof surface and prevents seeding of microbial microbes that may grow thereafter.

In this example, the volatile organic compound comprises ether. Since the ether evaporates relatively on contact from the system and dries the surface in box 26, a mammal's shoe surface with pre-applied adhesive thereon may immediately be placed on the bottom surface of the hoof to bond the shoe to the hoof without moisture therebetween. Moreover, there is less of a concern of growth of microbes on the hoof surface thereafter. As shown in box 28, the ether evaporates and forms a film-free coating of preferably an organic crystalline halogen salt on the surface. The film-free coating enhances bonding on the surface, since there is essentially no layer or coating between the hoof surface and the shoe surface. Thus, the film-free coating provides an increased surface area contact between bonding surfaces.

It is to be noted that the disinfectant system may be used as a pre-bonding application on other surfaces, organic or inorganic, without falling beyond the scope or spirit of present invention. For example, the system may be applied on metal surfaces for pre-bonding treatment.

In another example, the disinfectant may be used as a pre-treatment for wounds. In this example, a wound surface is washed, and the disinfectant system is applied thereon. The anti-microbial compound serves to disinfect the area of the wound and lessen the likelihood of seeding of microbial microbe growth thereafter. The carrier evaporates and leaves a film-free coating on the wound. The film-free coating allows for the wound to “breathe,” i.e., aerate or allow air to pass therethrough, thereby exposing the wound for healing. Moreover, a bandage may be immediately placed thereon, due to the evaporation of the carrier from the system which dries the wound substantially on contact.

Preferably, the wound is disinfected with the disinfectant system comprising the anti-microbial compound for disinfecting the surface. In this example, the anti-microbial comprises ethylenediamine dihydriodide. However, other anti-microbial compounds, each of which is compatible with the carrier, may be used. The wound is then immediately dried by the carrier of the disinfectant system. In this example, the carrier comprises the drying agent which preferably is an ether based compound, e.g., a mixture of between about 20% and 80% by weight methyl nonafluoroisobutyl ether and between about 20% and 80% by weight methyl nonafluorobutyl ether. The carrier evaporates from the system and leaves a film-free halogen salt coating on the surface. The film-free halogen salt coating allows for enhanced bandaging around the wound. The disinfectant system also allows for preparation on wounds prior to suturing.

While the present invention has been described in terms of preferred embodiments, it will be understood, of course, that the invention is not limited thereto since modifications may be made to those skilled in the art, particularly in light of the foregoing teachings.

1. A disinfectant system for drying a surface, the system comprising:
a carrier for drying the surface; and
an anti-microbial compound for disinfecting the surface, the anti-microbial compound having a halogen component to form a film-free coating comprising an organic halogen salt on the surface when the carrier evaporates from the surface.

2. The system of claim 1 wherein the carrier is a drying agent.

3. The system of claim 2 wherein the drying agent comprises an aerosol ether based solution.

4. The system of claim 1 wherein the carrier is an organic solution.

5. The system of claim 1 wherein the halogen compound is an organic iodine compound.

6. The system of claim 5 wherein the organic iodine compound includes ethylenediamine dihydriodide.

7. The system of claim 6 wherein the ethylenediamine dihydriodide is in a crystalline form.

8. The system of claim 1 wherein the carrier comprises between about 70 and 98 weight percent and the anti-microbial component comprises between about 2 and 30 weight percent of the system.

9. The system of claim 1 wherein the carrier comprises between about 80 and 95 weight percent and the anti-microbial component comprises between about 5 and 20 weight percent of the system.

10. A disinfectant system for preparing a surface for treatment, the system comprising:
an organic drying agent for drying the surface; and
an anti-microbial component for disinfecting the surface, the component having an organic iodine compound to form a film-free halogen salt coating on the surface when the drying agent evaporates from the surface.

11. The system of claim 10 wherein the organic drying agent is a mixture of between about 20% and 80% by weight methyl nonafluoroisobutyl ether and between about 20% and 80% by weight methyl nonafluorobutyl ether.

12. The system of claim 10 wherein the antimicrobial component comprises an organic iodine.

13. The system of claim 12 wherein the organic iodine comprises crystalline ethylenediamine dihydriodide.

14. A disinfectant system for preparing a hoof of a mammal prior for bonding, the system comprising:
a volatile organic compound for drying the surface;
an anti-microbial component for disinfecting the surface, the anti-microbial component containing ethylenediamine dihydriodide, forming a film-free halogen salt coating on the surface when the volatile organic component evaporates from the surface.

15. The system of claim 14 wherein the ethylenediamine dihydriodide is in the crystalline form.

16. A method of disinfecting a surface for drying, the method comprises:
applying a disinfectant system on the surface, the disinfectant system comprising a carrier for drying the surface and an anti-microbial component for disinfecting the surface, the anti-microbial component having a halogen compound to form a film-free coating comprising an organic halogen salt on the surface when the carrier evaporates from the surface.

17. The method of claim 16 wherein the carrier is a drying agent.

18. The method of claim 17 wherein the drying agent comprises a mixture of between about 20% and 80% by weight methyl nonafluoroisobutyl ether and between about 20% and 80% by weight methyl nonafluorobutyl ether.

19. The method of claim 16 wherein the carrier is an volatile organic solution.

20. The method of claim 16 wherein the antimicrobial component is an organic iodine compound.

21. The method of claim 20 wherein the organic iodine compound includes ethylenediamine dihydriodide.

22. The method of claim 21 wherein the ethylenediamine dihydriodide is in a crystalline form.

23. The method of claim 16 the carrier comprises between about 70 and 98 weight percent and the anti-microbial component comprises between about 2 and 30 weight percent of the system.

24. The method of claim 16 the carrier comprises between about 80 and 95 weight percent and the anti-microbial component comprises between about 5 and 20 weight percent of the system.

25. A method of preparing a surface for treatment, the method comprises:
applying a disinfectant system comprising an organic drying agent to dry the surface and an anti-microbial component to disinfect the surface, the anti-microbial compound comprising an organic iodine compound; and
forming a film-free halogen salt coating on the surface when the drying agent evaporates from the surface.

26. A method of preparing a hoof surface of a horse for bonding, the method comprising:
disinfecting the surface with a disinfectant system comprising a volatile organic component and an anti-microbial component, the anti-microbial component comprising ethylenediamine dihydriodide;
drying the surface with the volatile organic component; and
forming a film-free halogen salt coating on the surface when the volatile organic component evaporates from the surface, allowing for bonding on the hoof surface.
